(19) United States
(12) Patent Application Publication
(10) Pub. No.: US 2026/0076529 A1
(43) Pub. Date: Mar. 19, 2026

(54) AUGMENTED REALITY DISPLAY FOR A DENTAL TOOL

(71) Applicant: Onvi, Inc., Wilmette, IL (US)

(72) Inventor: Craig Kohler, Glenview, IL (US)

(21) Appl. No.: 19/109,490

(22) PCT Filed: Sep. 7, 2023
(86) PCT No.: PCT/US23/73634

§ 371 Date: Mar. 6, 2025

Related U.S. Application Data
(60) Provisional application No. 63/404,451, filed on Sep. 7, 2022.

(51) Int. Cl. A61B 1/00; A61B 1/04; A61B 1/24; A61B 5/00; A61C 9/00; G06T 7/00; G06T 7/70; G06T 7/90; G06T 11/00; G06T 13/00

(52) U.S. Cl. CPC A61B 1/0005; A61B 1/043; A61B 1/24; A61B 5/0071; A61B 5/0088; A61C 9/0053; G06T 7/0012; G06T 7/70; G06T 7/90; G06T 11/00; G06T 13/00; G06T 2207/10016; G06T 2207/10024; G06T 2207/30036; G06T 2210/41

(57) ABSTRACT

A dental tool system includes a dental tool having a camera, a UV light, and a working implement. The system also includes associated software that receives a video of the user's teeth captured by the dental tool camera, analyzes the video to determine a quantity of plaque on the plurality of teeth, superimposes icons at unclean locations so the icons are visible to the user in a substantially real-time display of the user's teeth and removes the icons after the user sufficiently brushes the unclean locations.

### CROSS-REFERENCE TO RELATED APPLICATION

This application claims the benefit of priority to U.S. application no. 63/404,451, filed on Sep. 7, 2022, and hereby incorporated by reference in its entirety.

### FIELD OF THE DISCLOSURE

This disclosure is generally directed to augmented reality for video from a medical instrument, such as a dental tool.

### BRIEF DESCRIPTION OF THE DRAWINGS

FIG. 1 is a block diagram view of an example video dental tool system.

FIG. 2 is a diagrammatic view of a portion of the example system of FIG. 1 in use.

FIG. 3 is a diagrammatic view of a display overlay that may find use with the system of FIG. 1.

FIG. 4 illustrates an example graphical user interface that may be used in conjunction with the system of FIG. 1.

FIG. 5 is a perspective view of a video toothbrush that may find use in the system of FIG. 1.

FIG. 6 is a perspective view of the video toothbrush of FIG. 5, with a portion of the case cut away to illustrate interior components.

FIG. 7 is a perspective view of portions of the system of FIG. 1, including an example video toothbrush.

FIG. 8 is a side view of the video toothbrush of FIG. 7, with a portion of the case shown as transparent to illustrate interior components.

FIG. 9 is a diagrammatic view of a light pipe that may find use for transmitting ultraviolet light in a video toothbrush.

FIG. 10 is a flow chart illustrating an example method of augmenting video from a dental tool.

FIG. 11 is a flow chart illustrating an example method of augmenting video from a dental tool.

FIG. 12 is a flow chart illustrating an example method of operating an ultraviolet light in the system of FIG. 1.

### DESCRIPTION

The instant inventor has appreciated that augmenting the display of video from a medical tool to provide information about a patient's teeth to patients and/or healthcare providers may be beneficial. For example, video from a dental tool may be augmented according to the instant disclosure. Such a video dental tool is illustrated and described in U.S. application Ser. No. 14/645,145, which is hereby incorporated by reference, and which is published as U.S. PG Pub. No. 2015/0257636. The remainder of this disclosure will refer to a video toothbrush for ease of description, but is not limited to such an embodiment. In addition, the remainder of this disclosure will refer to conditions of a patient's teeth, but it should be understood that other conditions in the user's mouth (e.g., canker sores) are within the scope and spirit of this disclosure, as are medical conditions associated with other portions of the body.

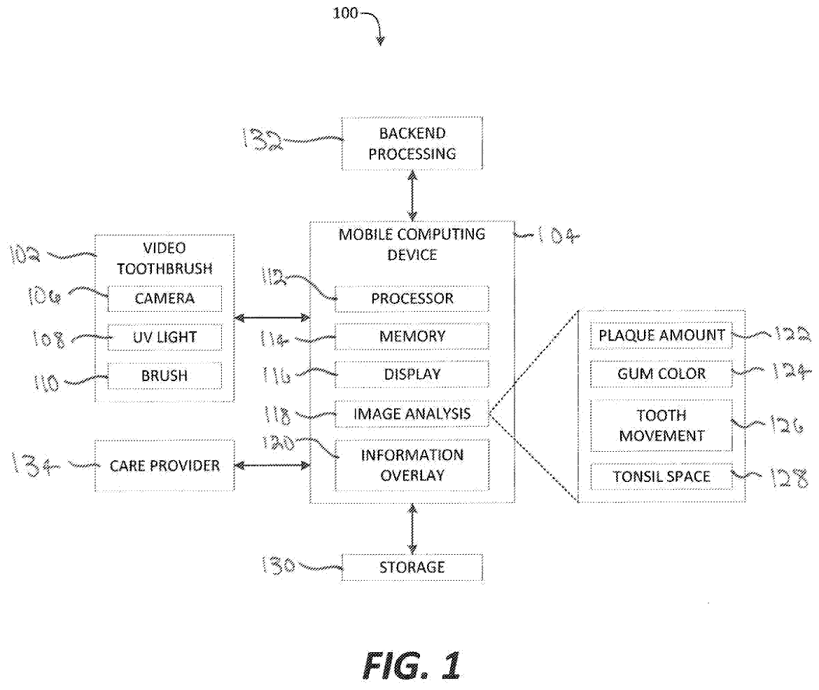

FIG. 1 is a diagrammatic view of a system 100 that includes a video toothbrush 102 and a mobile computing device 104. The video toothbrush 102 may capture include a camera 106, an ultraviolet (UV) light 108, and brush 110 or other working implement. The mobile computing device may include a processor 112 and a non-transitory, computer readable memory or other medium 114 storing instructions that, when executed by the processor 112, cause the mobile computing device 104 to perform one or more steps, operations, methods, processes, algorithms, etc. of this disclosure. The mobile computing device may further include a display 116 and one or more functional modules 118, 120 that may be embodied in hardware and/or software (e.g., as instructions in the memory 114).

The video toothbrush 102 may be used by a user to brush their teeth with the brush 110 while capturing video with the camera 106. The video toothbrush 102 may be in electronic communication with the mobile computing device 104 and may transmit the captured video feed to the mobile computing device 104. In turn, the mobile computing device 104 may display the video feed on the display 116 substantially in real-time. As will be described below, the mobile computing device 104 may also analyze and/or augment the video stream.

The functional modules 118, 120 may include an image analysis module 118 which may receive the video stream and analyze the video to determine if one or more conditions are present. For example, the image analysis module 118 may include a plaque amount sub-module 122 that may determine the presence and location of plaque on teeth, a gum color sub-module 124 that may determine the color and change in color of the user's gums at one or more locations, a tooth movement sub-module 126 that may determine locations and relative movement of teeth, and a tonsil space sub-module 128 that may determine the size and change in size of the user's upper airway, and/or other sub-modules for other medically-relevant or informational determinations.

The plaque amount sub-module 122 may analyze images in a real-time video feed to determine an amount of plaque and location of plaque. For example, the plaque amount sub-module 122 may perform processing on images following UV exposure and analyze those images, as described with respect to FIGS. 10, 11, and 12.

The gum color sub-module 124 may take a number of sample images of the user's mouth from a given video feed and determine an average gum tissue color associated with that video session (e.g., brushing session). The gum color sub-module 124 may store that average gum color and may compare that average gum tissue color to one or more previously-stored gum tissue colors for the user. If the gum color sub-module 124 determines, through that comparison, that the user's gum tissue color has changed (e.g., deviates from a previous color) by more than a threshold amount, the gum color sub-module 124 may cause a notification to be sent to the user that the user's gum color has changed, and/or that the user may be at risk for gum disease.

The tooth movement sub-module 126 may determine and store the location of each of the user's teeth during a brushing session. Tooth locations may be determined with respect to other teeth, for example. The tooth movement sub-module 126 may compare the location of each tooth to previously-stored locations of the user's teeth. If the tooth movement sub-module 126 determines, through that comparison, that one or more of the user's teeth has moved by more than a threshold amount, the tooth movement sub-module 126 may cause a notification to be sent to the user that the user's tooth or teeth has or have moved.

The tonsil space sub-module 128 may determine a contour of the user's upper airway, including the size and protrusion of the user's tonsils, based on video captured by the video toothbrush 102. The contour of the user's upper airway may be compared to known contour features (e.g., oversized tonsils, undersized airway space) that are indicative of the onset of sleep apnea or other respiratory issues. If the tonsil space sub-module 128 determines, through that comparison, that the user's upper airway contour is consistent with a potential respiratory issues, the tonsil space sub-module 128 may cause a notification to be sent to the user that the user's upper airway is consistent with a potential respiratory problem.

The functional modules 118, 120 may further include an information overlay module 120 that may overlay one or more items of information, graphics, etc. to augment the video feed displayed on the display 116 based on determinations by the image analysis module 118. For example, the information overlay module 120 may add one or more icons on one or more respective locations on the user's teeth on which plaque is determined to be present, to teeth that are determined to have moved, to gums that have discolored, etc.

The mobile computing device 104 may further be in electronic communication with external data storage 130, such as cloud storage. For example, the external data storage 130 may be associated with an application executing on the mobile computing device 104, which application may include the modules 118, 120 and which may output the video feed on the display 116. The mobile computing device 104 may transmit video feeds (in original form and/or augmented form), determinations made by the image analysis module 118, etc. to the storage 130 and may retrieve the same from storage 130. Additionally or alternatively, the mobile computing device 104 may store video feeds, determinations, and/or other information in the memory 114.

The UV light 108 of the video toothbrush 102 may be used to identify plaque on the user's teeth. For example, the user may activate the UV light 108, shine the UV light on one or more portions of the user's mouth, and then deactivate the UV light 108. In some embodiments, the UV light 108 may be deactivated by the video toothbrush 102 automatically after a predetermined amount of time or in response to the user's mouth being sufficiently exposed to UV light based on video analysis. Because plaque is readily identifiable in video after UV exposure, the image analysis module 118 may identify plaque that has been exposed to UV light. In some embodiments, the video toothbrush may further include a permanent or retractable filter over the camera 106, which filter may perform a color correction function for identifying clean teeth as white and plaque as red in color, after that plaque is irradiated with UV light.

The UV light 108 may emit light across the UV spectrum, in embodiments. For example, the UV light 108 may be a UV curing light that outputs light in the range of 385-490 nm. In some embodiments, the UV light 108 may emit light at a frequency centered on a frequency that is particularly useful for plaque irradiation and identification, such as 405 nm.

In addition to or instead of local execution of the modules 118, 120 on the mobile computing device 104, a backend processing source 132 (e.g., a cloud service) may execute one or more aspects of the image analysis module and/or information overlay module. Accordingly, the mobile computing device 104 may be in electronic communication with the backend processing 132 to, e.g., transmit raw and/or augmented video between the backend processing 132 and the mobile computing device 104, transmit image analysis-based determinations between the backend processing 132 and the mobile computing device 104, etc.

The mobile computing device 104, data storage 130, and/or backend processing 132 may further be in electronic communication with one or more care providers 134. For example, the mobile computing device and/or backend processing 132 may transmit image analysis-based determinations to the care provider (e.g., dentist or other dental health professional) with appropriate consent from the user. Any notification disclosed herein as being transmitted to the user may, instead or in addition, be transmitted to a care provider 134. Further, the care provider may, with appropriate consent from the user, access the user's stored videos or determinations in the storage 130, send comments to the user based on the care provider's review of videos and/or determinations, etc.

In some embodiments, the video toothbrush 102 may be in electronic communication with one or more both of the backend processing 132 and data storage 130 independent of the mobile computing device 104. Accordingly, the video toothbrush 102 may transmit video to and/or receive instructions from the backend processing 132 and/or data storage 130 in addition to or instead of similar communications with the mobile computing device 104.

The video toothbrush 102 may record video with the camera 106 that is included in or mounted on the toothbrush 102, and may output that video to one or more displays or storage devices (e.g., external storage 130 or memory 114). In an embodiment, the video from the toothbrush may be output to and displayed on the mobile computing device 104, which may be a smartphone, tablet, or other device. Additionally or alternatively, the video may be output to and displayed on a user-wearable device, such as a smartwatch, smart glasses, and the like. Additionally or alternatively, the video may be output to and displayed on a hologram projector. Accordingly, output on a display of the video from the toothbrush may be substantially in real-time, or may be a substantial time after the video was recorded (e.g., replay or stored video).

The video toothbrush 102 may be used by a consumer, in an embodiment, as part of their regular hygiene routine. Additionally or alternatively, the video toothbrush 102 may be used by a dental professional as part of treating patients.

A video captured by a video toothbrush 102 may be viewed by the user of the toothbrush 102 (e.g. the computing device 104 or wearable device of a consumer when the toothbrush is used by the consumer, or a display associated with a dental professional, when the toothbrush is used by a dental professional), in an embodiment. Additionally or alternatively, the video may be viewed by a third party that received the video, or permission to view the video, from the capturer. For example, such a third party may be a dental professional that received the video from a patient.

Various information may be used to augment the display of the video captured by the video toothbrush 102. For example, such information may be overlaid on or presented adjacent to the video. Several example modes of augmentation are discussed in turn below. The various modes will be described with reference to the mouth of a “patient.” It should be understood that this is for ease of description only, and the various modes may find use by either a dental or medical professional or a consumer. These modes may be implemented via the functional modules 118, 120, in embodiments.

In one or more modes of use, the video may be augmented by overlaying information about conditions of the patient's teeth. For example, known fillings, crowns, implants, etc. may be color-coded in the video, labeled with text, or identified in some additional or alternative way. Such information may be based on clinical information provided by a medical care provider, input from a consumer/patient, or another source outside of the video, in an embodiment. Additionally or alternatively, such information may be determined by analysis of the video itself. For example, the video may be analyzed to identify and tag the patient's teeth and conditions associated with those teeth, such as a computer processor of a user mobile electronic device. Such video analysis, like all video analysis in this disclosure, may be performed by the mobile computing device 104, processing capacity of the video toothbrush 102, and/or by backend processing 134.

In one or more modes of use, the video captured by the video toothbrush 102 may be used as a basis to create a model of a patient's teeth or other body part. For example, a video captured by the toothbrush 102 may be used to create a partial or complete three-dimensional model of the patient's teeth. Accordingly, the video may be analyzed to create a model. In any of the below-described modes of use, a video of the patient's teeth may be overlaid on such a model, displayed next to the model, or the model may be displayed instead of the actual video feed, based on the field of view and position of the camera. Accordingly, while the modes of use below will be described with reference to displaying information with or on the video, such information may additionally or alternatively be displayed on or with a model. In addition to or instead of a model of the patient's teeth, one or more icons or other graphical features may be overlaid on the actual images of the user's teeth, as will be described below.

In a “live mode,” the video may be supplemented to show a wider field of view than is actually captured by the camera 106. For example, in an embodiment in which the live video would normally display three or four teeth based on the position of the camera 106, field of view of the camera 106, etc., the display of the video may augment the teeth actually captured in the video with representations of the surrounding teeth in the patient's mouth (e.g., models that appear to the user to be the teeth themselves). Icons and other image augmentation according to this disclosure may be overlaid or added to such models as well as to the teeth within the field of view of the camera 106. Additionally or alternatively, “live mode” may augment the display by allowing the user to zoom in on a particular portion of the video frame, such as by holding the camera lens in a single location for a given period of time. Additionally or alternatively, the “live mode” may zoom in by default, offering a magnified view of the patient's teeth.

In “x-ray mode,” the video may be augmented by overlaying x-rays of a user's teeth on the video or displaying x-rays along with the video. The x-rays may be 2D or 3D x-rays, in embodiments. In other embodiments, images from other imaging modalities may be overlaid or displayed along with the video. Icon and other image augmentation according to this disclosure may be overlaid or added to such x-ray images as well as to the actual video feed from the camera 106.

In “mobility mode,” the toothbrush (or other instrument) may be used to apply pressure to a tooth or other body part, and the video may be displayed so as to note movement of the tooth (e.g., by displaying an “original” and a “moved” position of the tooth). Additionally or alternatively, the video may be displayed so as to note movement of one or more teeth relative to a previously-captured video (e.g., as described above with reference to the tooth movement sub-module 126).

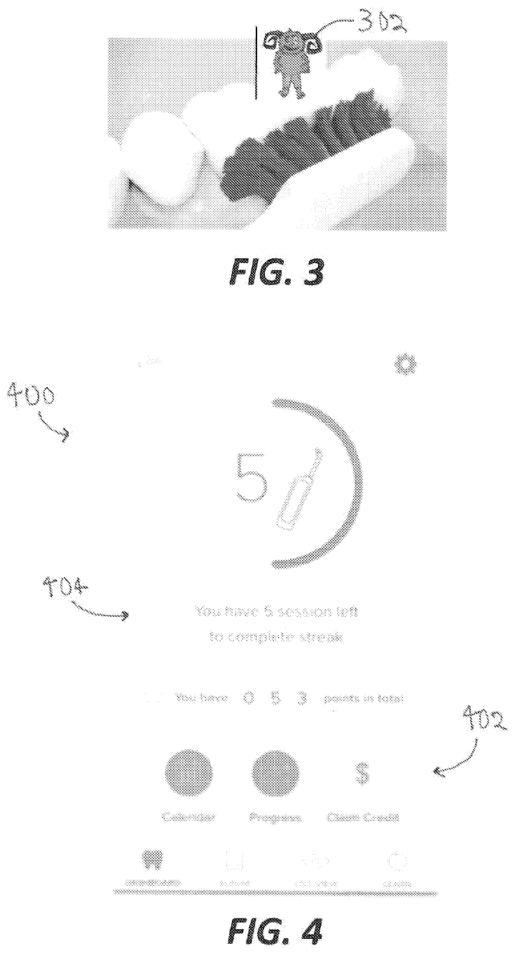

In “plaque mode,” the video may be overlaid with information about plaque on the patient's teeth. For example, plaque may be color-coded on the video to provide the user with an indication of specific areas to be cleaned with the brush. “Plaque mode” may include the use of a UV light on the toothbrush to show the plaque, in an embodiment. FIGS. 3 and 9-12 illustrate various aspects of “plaque mode,” in which “monsters” or other illustrations are superimposed on areas of teeth having plaque, so that the user can “attack” the monsters by brushing the plaque-covered area. In response to the plaque being cleared from a tooth, the overlaid monster or other illustration may be removed.

In “clinical mode,” the video may be overlaid with or displayed with information for performing a clinical procedure. For example, a location to be drilled, filled, incised, lasered, or otherwise treated may be color-coded or otherwise annotated for a clinician to view.

The video may also be used to determine a patient's ability to home-treat a condition, or success in an attempt to home-treat a condition. For example, in the event that a patient is not able to sufficiently clean plaque at home or treat another condition identified in the video, the video may be transmitted to a care provider, such as the patient's dentist, for assistance with the condition (e.g., an explanation of the condition, a recommendation for treatment, to schedule an appointment, etc.).

The video and tagged information may be used to recommend further treatment. For example, where a patient's teeth have changed such that a negative condition appears to be forming (e.g., an incipient cavity, excessive tooth wear, etc.), recommendations may be provided to the patient automatically (e.g., a recommendation to schedule a fluoride treatment, a recommendation to brush a particular area with greater care, etc.). Such recommendations may be determined by a processor of the patient's mobile computing device, for example.

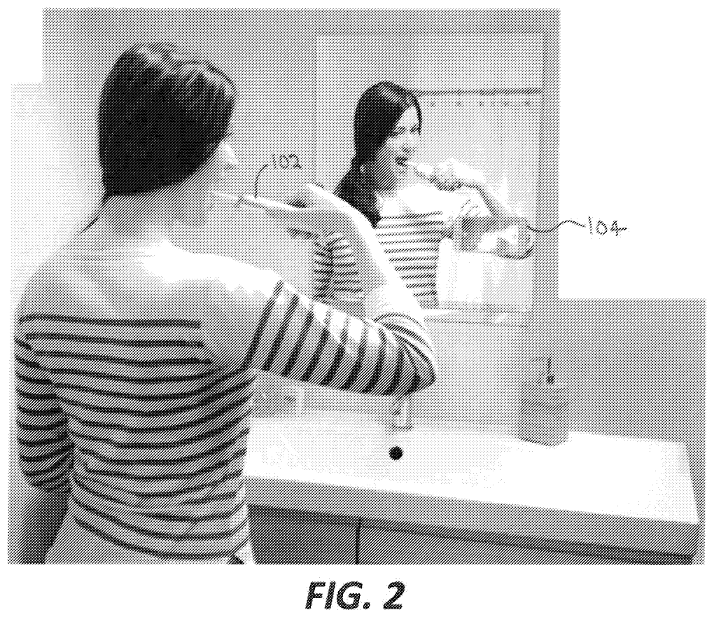

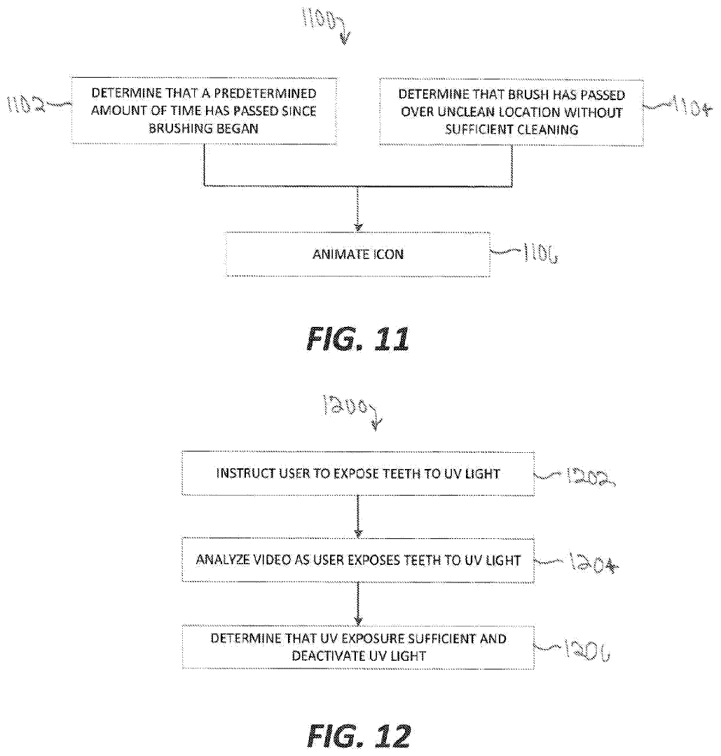

FIG. 2 is a diagrammatic view of use of the system 100. As shown in FIG. 2, the toothbrush 102 may capture video of the user's mouth, including the user's teeth, while the user brushes their teeth. The toothbrush 102 may stream live video to the user's mobile computing device 104, which may display the live video of the user's mouth for the user to view while brushing. Referring to FIGS. 1-3, the image analysis and information overlay modules 118, 120 may determine an amount of plaque on the user's teeth or other information and overlay one or more icons 302 on the user's teeth based on those determinations. For example, icons 302 may be overlaid in the live video where the user's teeth have above a threshold amount of plaque. The icons 302 may be animated or otherwise altered based on user brushing interaction with the icons 302, as discussed with respect to FIGS. 10 and 11. Further, the icons may be placed based on an amount of plaque on the user's teeth, which may be determined in the first instance based on images captured by the video toothbrush 102 after exposure of UV light on the user's teeth, which UV light may also be emitted by the video toothbrush 102.

FIG. 4 illustrates an example user interface portion 400 that may be presented on the user's mobile computing device, such as in a portion of an application that also displays the live video feed from the toothbrush 102 in another portion. The user interface portion 400 may include a plurality of user-selectable menu items 402, including a calendar menu item, a progress menu item, a claim credit menu item, a dashboard menu item, a live view menu item, and a learn menu item. The user interface portion may enable the user to select from among the various modes described above. The user interface portion 400 may report user participation in one or more activities, such as a day-by-day brushing streak, for example, and/or a score for a particular time period, in a reporting portion 404. Both the score and the brushing streak may be determined by the application based on user interaction with icons. For example, a user may add a day to the streak by having a brushing session in which all icons are brushed based on the associated plaque being removed. In another example, a user may add a day to the streak by brushing twice in the day with a certain number of icons brushed. Other information discussed herein, such as notifications based on analysis of video of the user's mouth, may also be presented to the user in interface portion 400. Additionally or alternatively, such notifications may be transmitted to the user via email, text message, etc.

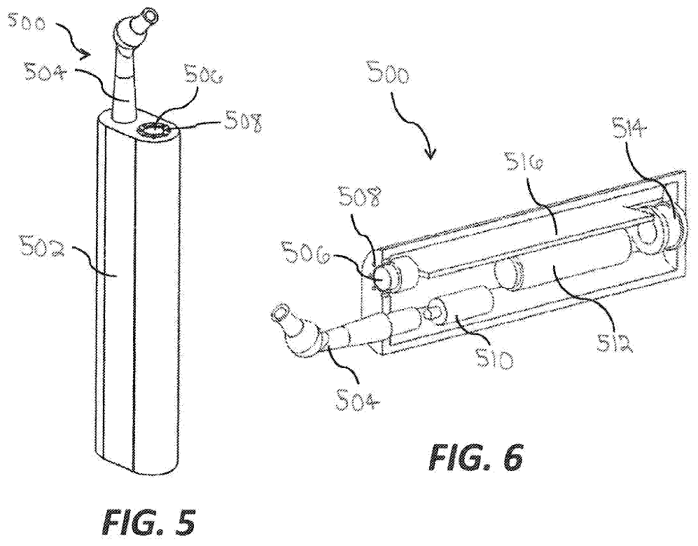

FIGS. 5-8 illustrate various example features of a video toothbrush. First, FIGS. 5 and 6 illustrate a dental instrument 500 (which may serve as the video toothbrush 102) including a body 502 and a dental instrument insert 504 extending from an end of the body 502 for use on a patient's oral cavity. Disposed adjacent the dental instrument insert 504 may be a camera lens 506. The camera lens 506 may allow an image or a video to be recorded, and may preferably have a line of sight that is roughly parallel with the dental instrument insert 504 extending from the body 502 of the dental instrument 500 and may be aimed at the working tip of the dental instrument insert 504. However, the camera lens 506 may further have a relatively wide angle to record or show a relatively large viewing field. Thus, the dental instrument 500 may display images and/or video of the working tip of the dental instrument insert 504 and, especially, during use of the same in a patient's oral cavity. In a preferred embodiment, the dental instrument 500 may have a plurality of lights 508, such as, for example, LEDs that may be ring the lens 506 and provide a sufficient light source for the recording and/or viewing of images and/or video. In another embodiment, the lights may be ultraviolet lights or a combination of visible and ultraviolet lights that may aid a user of the dental instrument 500 in viewing plaque on a patient's tooth, or for curing epoxies and the like within a patient's oral cavity.

FIG. 6 illustrates a cut-away side view of the dental instrument 500, in accordance with the present invention. Specifically, the dental instrument 500 may comprise the aforementioned dental instrument insert 504 that may preferably be removable from the body 502 so as to be replaceable with other dental instrument inserts, such as a pick, a scaler, a flosser, etc. The dental instrument inset 504 may be placed within an aperture in the body 502, and when disposed therein may be mechanically tied to a motor and transmission 510, which may be electrically tied to a battery/power source 512 within the body 502. The dental instrument 500 may be powered via the battery/power source 512, and may preferably be rechargeable via charging element 514, which may be electrically coupled with a power source for charging the battery/power source 512, as needed.

The lens 506 and lights 508 may be disposed on an end of the body on the same end as the dental instrument insert 504, such that the camera lens 506 and lights 508 may facilitate the recording and/or viewing of the dental instrument insert 504 when in use within a patient's oral cavity. Coupled to the lens 506 and lights 508 may be a main PCB board 516 that may control the recording and viewing of the images and/or video through the lens 506, and may further control the lights 508. The main PCB board 516 may allow a user to turn on or off the lens 506 for the recording and/or viewing of the same, or may allow a user to turn on or off the lights 508, as needed.

Thus, when in use, a user may insert a dental instrument insert 504 into the aperture of the body 502 of the dental instrument 500. The dental instrument 500 may have previously been coupled to a display device (not shown), either wired or, preferably, wirelessly, for viewing and/or recording images and/or video via lens 506. The user may have the ability to control the turning on or off of the lens 506 and/or the lights 508 via the display device (not shown), via the body 502, or via any other means or mechanism to control the same.

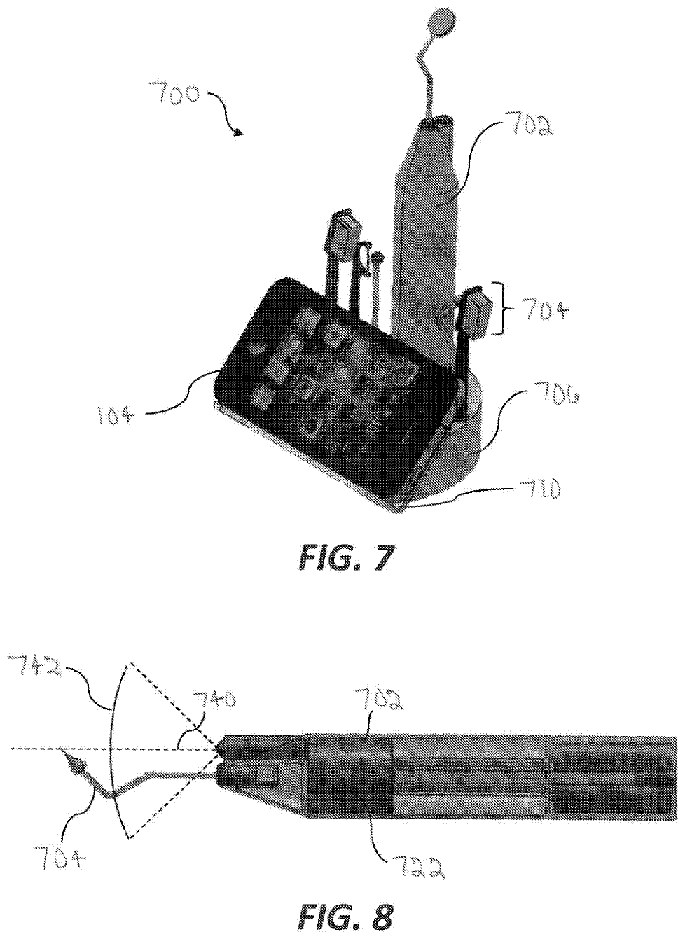

FIG. 7 illustrates a system 700 that includes a dental tool 702 (which may serve as the video toothbrush 102), a plurality of dental tool instrument tool inserts 704, a base 706, and a display device (e.g., user mobile computing device) 104 for displaying video thereon.

The base 706 may hold the dental tool 702, the plurality of tool inserts 704, and may further hold the display device 104. Specifically, the base 706 may operate as a charging cradle for holding and/or charging the dental tool instrument 702 and/or the display device 104. More specifically, the base 706 may comprise a plurality of apertures or cradles for holding the dental tool 702 and the plurality of dental tool inserts 704.

The display device 104 may rest on or within a platform 710. The platform 710 may be angled so as to allow the display device 104 to be viewable by a user of the dental tool instrument 702 and/or a patient. The platform 710 may be adjustable so as to tilt or otherwise reposition the display device 104 for better view thereon, and may have a frame, flanges or other like holders for holding the display device 104 thereon or therein.

The display device 104 may preferably be a smart phone, such as an iPhone®, an Android® phone, a tablet computer, such as an iPad®, or other like display device that allows streaming video to be wirelessly sent from the dental tool 702, as described in more detail below. The display device 104 may preferably receive the wireless streaming video from the dental tool 702 via any manner apparent to one of ordinary skill in the art, such as via WiFi, 3G cellular telephone networks, Bluetooth®, or other like data transmission protocol, and via any video codec, such as MPEG4, M-JPEG or other like video codec.

The plurality of dental tool inserts 704 may be any dental tool instruments that may be useful for a dental practitioner to clean, repair, or otherwise tend to a patient's oral cavity. Common dental tool instruments may include brushes, scalers, mirrors, probes, syringes, drills, burs, excavators, burnishers, excavators, elevators, forceps, curettes, and any other like instrument that may be usefully employed from the dental tool instrument 702. In addition, it should be noted that the present invention may allow a plurality of instruments to be utilized at the same time, such as, for example, a dental mirror and a scaler to aid in the use of the scaler.

FIG. 8 illustrates a cut-away perspective view of the dental tool 702. The dental tool 702 may generally comprise a housing 720 in which may be contained a camera 722 having a lens 724 for shooting video and/or still photographs, one or more processing boards 726, a rechargeable battery 728, a power cable 730, a charging input means 732, and a dental instrument insert aperture 734 for holding one or a plurality of dental instrument inserts 704. One or more light sources (not shown) may further be provided for directing illumination at the dental instrument insert 704 and/or the patient's oral cavity. The housing 720 may be sealed and made of a material resistant to a moist environment, such as a metal or plastic, as apparent to one of ordinary skill in the art.

In use, a user, such as a dental practitioner, may insert a dental instrument insert 704 into the dental instrument aperture 734, which may securely hold the dental instrument therein, such as via clamping means, frictional resistance means, or the like. The camera lens 724 is positioned on an end of the housing 720 and directed toward the tip of the dental instrument insert 704, providing a relatively wide viewing cone 742 for seeing the patient's oral cavity and the dental tool used within the patient's oral cavity. Specifically, the lens 724 may be adjacent the shaft of the dental instrument insert 704 and may provide a line-of-sight 740 for the camera that is roughly parallel with the shaft of the dental instrument insert 704. Thus, the dental instrument insert 704 and, specifically, the working tip of the dental instrument insert 704, may be easily viewable by the user of the dental tool 702 and/or the patient via the camera 722. In a preferred embodiment, the lens 724 may be aimed directly at the working tip of the dental instrument insert 704 for specific and precise viewing of the working tip of the dental instrument insert 704. One or more light sources, such as white LEDs, or the like, may further be used to illuminate the dental instrument insert 704 and/or the patient's oral cavity.

In a preferred embodiment, a dental mirror may be utilized to allow the camera 722 to record video and/or still photographs of an area that is ninety degrees, or any other angle, to the line-of-sight 740 of the camera 722, such as within a patient's oral cavity. Moreover, an illumination source emanating from the dental tool 702 may further be reflected off the dental mirror to aid in illuminating a patient's oral cavity.

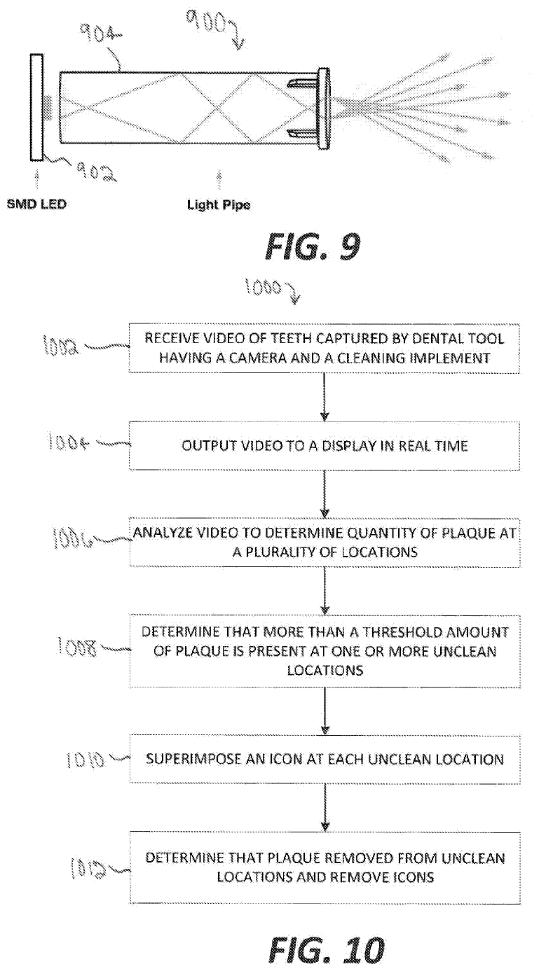

In some embodiments, a video toothbrush 102 may include a UV light for illuminating a user's oral cavity so as to reveal plaque on the user's teeth, for example. FIG. 9 illustrates an example UV light arrangement 900 for including a UV light in a video toothbrush 102 according to the present disclosure. The UV light arrangement 900 includes a UV surface mounted device (“SMD”) LED 902 or other LED capable of emitting light in the UV frequency and a light pipe 904 that transmits the output light from the LED 902 to a desired output position and angle on the video toothbrush. For example, the UV light output may be proximate a camera lens (e.g., as illustrated for the lights 508 in FIG. 5). Alternatively, a UV LED may be mounted proximate its output with a lens to create an appropriate output angle and pattern, and white light or other visible light LEDs may also be placed in the same video toothbrush 102, so that the video toothbrush 102 can selectively output visible light and/or UV.

FIG. 10 is a flow chart illustrating an example method 1000 of operating a video toothbrush system. The method 1000, or one or more aspects of the method 1000, may be performed by one or more components of the system 100, in embodiments. For example, one or more of the video toothbrush 102, the user mobile computing device 104, and the backend processing 132 may perform one or more of the operations of the method 1000.

The method 1000 may include, at operation 1002, receiving a video of teeth captured by a dental tool having a camera and a cleaning implement. The cleaning implement may be within the field of view of the camera. The video may be received by the dental tool itself (e.g., via its camera) and/or may be received by a user mobile computing device, backend processing, or other processing source.

The method 1000 may further include, at operation 1004, outputting the received video to a display in real time. The received video may be output, for example, on a display of a user mobile computing device, to a projector, to a pair of VR goggles, etc. or to any other appropriate display device. The real-time output may continue while further operations described below are performed, in some embodiments.

The method 1000 may further include, at operation 1006, analyzing the received video to determine a quantity of plaque at a plurality of locations. Dental plaque can be identified by the light-induced fluorescence (LIF) technique, which is based on the red fluorescence property of porphyrins, metabolites of heterogeneous bacteria within dental plaque, when irradiated with narrow blue-violet light (e.g., centered at 405 nm wavelength).

Operation 1006 may include, in some embodiments, a multi-stage process for identifying plaque in images. First, object detection may be performed on each image frame for determining whether the input image is an oral image and localizing the oral region. Second, instance segmentation may be performed for extracting the plaque regions.

In object detection, a ground-truth bounding box technique may be applied to outline the region of interest involving only the teeth and gums for each image may be identified, then a more fine-tuned bounding box detector may be applied to detect the RoI within the images (predicted bounding box).

In instance segmentation, in order to finely extract the red fluorescence-emitted dental plaque regions (e.g., a plurality of regions) from the oral image, a machine learning model may be applied for identifying red fluorescence on individual teeth, as well as red fluorescence that spans multiple teeth or that is between teeth, to determine where corresponding plaque is on the teeth. The model may be or may include, for example, a recurrent neural network trained on a set of red fluorescence images. Details of an example model are provided in Kim et al., Light-Induced Fluorescence-Based Device and Hybrid Mobile App for Oral Hygiene Management at Home: Development and Usability Study, JMIR Mhealth Uhealth. 2020 Oct. 16; 8(10):e17881. doi: 10.2196/17881. PMID: 33064097; PMCID: PMC7600004.

Operation 1006 may include identifying the width and height of areas of plaque on individual teeth, as well as plaque between teeth and areas of plaque that span multiple teeth. Accordingly, operation 1006 may include determining the size and approximate shape of each plaque region.

The method 1000 may further include, at operation 1008, determining that more than a threshold amount of plaque is present at one or more unclean locations on the user's teeth. In some embodiments, a certain size of a particular region of plaque may be used as a threshold (e.g., where plaque regions below that size are considered “clean” or are not flagged for further cleaning). Such a size threshold may be a square area, a width, and/or a height. Additionally or alternatively, the threshold amount of plaque may be determined according to a total amount of fluorescence associated with a region of plaque. Such a total fluorescence may be, for example, the cumulative intensity of red pixels in a plaque area. More generally, the threshold may be based on a size of a plaque region or a total intensity in an image of a plaque area. Plaque regions that exceed the threshold may be considered “unclean.”

The method 1000 may further include, at operation 1010, superimposing an icon at each unclean location. The icon may be an appropriate image to call the plaque area to the user's attention and motivate the user to brush the plaque region. For example, the icon may be a monster (as shown in FIG. 3) or other anthropomorphic image, a simple shape (e.g., square, circle, etc.), an arrow, or any other icon visible to the user. The icon may be superimposed on the live video frame at the locations determined to be unclean. The icons may be superimposed such that, as the user moves the camera and changes the field of view and related video, the icons may move along with the field of view or viewing frame so that the icons remain displayed and superimposed on the unclean locations on the user's teeth.

The method 1000 may further include, at operation 1012, determining that the plaque has been removed from the unclean locations and removing the icons. Operation 1012 may include the same or similar processes as operation 1006 and determining that the amount of plaque at a location that was previously (at a first time) determined to be “unclean” at operation 1008 is now (at a second time) clean, i.e., the amount of plaque no longer exceeds the threshold applied at operation 1008. In response, the icon may be removed from the video view.

In some embodiments, operation 1012 may include altering an icon as a user brushes on or proximate to the location on which the icon is superimposed. For example, an icon may be animated as the associated unclean location is being brushed, as visual feedback to the user that the correct location is being brushed. In another example, the size, brightness, or other characteristic of the icon may be altered to indicate a reduction in the plaque amount or the presence of the toothbrush at the location of the plaque.

As the user brushes—during which the user is both moving the camera and removing plaque—operations 1002, 1004, 1006, 1008, 1010 and 1012 may be continuously performed as to each frame, or a predetermined subset of frames (e.g., one frame per second, two frames per second, etc.) in the live video feed so that plaque locations are continuously annotated with icons, and those icons are altered or removed as the user brushes to provide the user with real-time feedback on their brushing progress. In some embodiments, the method 1000 may additionally include analyzing the video to determine a gum color deviation, potential upper respiratory contour indicative of a respiratory problem, tooth movement, and/or other physiological condition that can be determined from video, as discussed above. Where such analyses are indicative of a potential problem, the method 1000 may further include transmitting a notification to the user, as discussed with respect to FIG. 1. Still further, in some embodiments, the method 1000 may further include receiving contact information for a care provider (e.g., from the user or from the care provider), receiving a user's consent to share diagnostic information with the care provider, and sharing one or more analyses, notifications, images, videos, conclusions, etc. of this disclosure with the care provider.

FIG. 11 is a flow chart illustrating an example method 1100 of operating an augmented reality output. The method 1100, or one or more aspects of the method 1100, may be performed by one or more components of the system 100, in embodiments. For example, one or more of the video toothbrush 102, the user mobile computing device 104, and the backend processing 132 may perform one or more of the operations of the method 1100.

The method 1100 may include various non-cleaning conditions and scenarios in which a superimposed icon may be animated so as to draw the user's attention to the icon and cause the user to brush the plaque associated with the icon. In addition to the example reasons and scenarios given in method 1100, an icon may be animated in numerous other situations consistent with this disclosure.

The method 1100 may include, at operation 1102, determining that a predetermined amount of time has passed since brushing began, i.e., that a predetermined passage of time has occurred. That amount of time may be, for example, one minute, ninety seconds, or two minutes since brushing began for the whole mouth. Additionally or alternatively, the amount of time may be 15 seconds, 20 seconds, or 30 seconds since a user began brushing a quadrant of their mouth.

The method 1100 may further include, at operation 1104, determining that the brush has passed over an unclean location without sufficiently cleaning the unclean location, i.e., without sufficiently removing the plaque. Operation 1104 may include, for example, determining that an amount of plaque (e.g., a quantity of red fluorescence) has reduced, but has not reduced below a threshold for classifying the location as “clean.” Additionally or alternatively, operation 1104 may include determining that the brush has coincided with the location at which plaque is known to exist (from having been annotated with an icon previously), but that an amount of plaque remains above the threshold to classify the location as “unclean.”The method 1100 may further include, at operation 1106, animating the icon in response to either operation 1102 or operation 1104 (that is, in response to determining that a non-cleaning condition has been met). Animating the icon may include, for example, causing the icon to move (e.g., rotate), causing the icon to become a multi-frame “video” icon, such that the icon appears to dance, wave, wiggle, etc., causing the icon to pulse, causing the icon to change size or shape, etc. In short, operation 1106 may include changing the appearance of the icon in response to operations 1102 or 1104. Thus, as the plaque is removed to smaller amounts the icons may change in real time (e.g., monster does a death flip, gets wounded, slouches to a death scream, etc.). The visual representation can change to a smaller monster or other representation if all the plaque is not removed. It is possible that incipient decay or decalcification of enamel may be under the plaque. This area of possible decay may be marked as something else (igloo, stop sign etc.) for the dentist to investigate.

FIG. 12 is a flow chart illustrating an example method 1200 of exposing a user's teeth to UV light to assess the presence of plaque. The method 1200, or one or more aspects of the method 1200, may be performed by one or more components of the system 100, in embodiments. For example, one or more of the video toothbrush 102, the user mobile computing device 104, and the backend processing 132 may perform one or more of the operations of the method 1200.

The method 1200 may include, at operation 1202, instructing the user to expose their teeth to UV light. The instruction may be made via a display, such as a display of a user mobile computing device. Alternatively, the instruction may be made via a video toothbrush, such as by an output sound, a recorded verbal output instruction, an output light pattern, etc. The UV light may be disposed on a video toothbrush, and thus the video toothbrush may capture video of the user's mouth as it is exposed to UV light.

The method 1200 may further include, at operation 1202, analyzing an output video from the video toothbrush as the user exposes their teeth to UV light. Operation 1202 may include, for example, analyzing exposure of a plurality of surfaces within the user's mouth to determine if UV light has been applied to one or more teeth. In some embodiments, operation 1202 may include assessing the UV exposure of teeth individually and adding or removing tags on teeth as those teeth are sufficiently exposed to UV light. Sufficient exposure of a tooth may be determined by, for example, determining a duration of exposure for the tooth based on images captured by the camera that include UV light exposure, based on a duration of the tooth being in a portion of the video frame that correlates to the emission field of the UV light, etc.

The method 1200 may further include, at operation 1204, determining that the UV exposure is sufficient and, in response, automatically deactivating the UV light. Automatic deactivation may serve as a safety measure to prevent intentional or unintentional overexposure of the user to UV light. UV exposure may be determined to be sufficient when each tooth has been exposed sufficiently, when a certain percentage of teeth have been exposed sufficiently, or when a total exposure time has reached a threshold, for example.

In some embodiments, the methods 1000, 1100, 1200 may be used in conjunction. For example, method 1200 may be performed to expose the user's teeth to UV light to enable plaque identification, then method 1000 may be performed for the user to clean the plaque. During the performance of method 1000, method 1100 may also be performed to add further detail and interest to the augmented reality display.

In a first aspect of the present disclosure, a method for augmenting video from a dental tool is provided. The method includes receiving a video of a plurality of teeth, the video captured by a dental tool comprising a camera and a cleaning implement, causing the video to be output to a display visible to a user of the dental tool substantially in real time, analyzing the video to determine a quantity of plaque at a plurality of locations on the plurality of teeth, determining, at a first time, that the quantity of plaque exceeds a threshold for an unclean location of the plurality of locations, superimposing a respective icon in the video at the unclean location such that the respective icon is visible to the user when the unclean location is visible to the user in the substantially real-time output, and determining, at a second time, that the quantity of plaque at the unclean location no longer exceeds the threshold and, in response, removing the icon from the superimposed video.

In an embodiment of the first aspect, the method further includes determining that a non-cleaning condition for an unclean location has been met and, in response, causing the icon associated with the unclean location to animate. In a further embodiment, the non-cleaning condition includes a predetermined passage of time after the first time, or a passage of a working implement of the dental tool over the unclean location without sufficient removal of plaque.

In an embodiment of the first aspect, analyzing the video to determine a quantity of plaque at a plurality of locations on the plurality of teeth comprises detecting red fluorescence at each of the plurality of locations.

In an embodiment of the first aspect, the icon is a first icon, and removing the first icon from the superimposed video comprises replacing the first icon with a second icon.

In an embodiment of the first aspect, analyzing the video to determine that a working implement of the dental tool is at the unclean location and, in response, animating the icon.

In an embodiment of the first aspect, the method further includes outputting an instruction for a user to expose the user's teeth to a UV light of the dental tool.

In a second aspect of the present disclosure, a dental tool system is provided that includes a dental tool having a camera, a UV light, and a working implement. The system further includes a non-transitory, computer readable medium storing instructions that, when executed by a processor of a computing system, cause the computing system to perform operations including instructing a user, via a display, to irradiate the user's teeth using the UV light, receiving a video of a plurality of teeth, the video captured by the dental tool camera, causing the video to be output to a display visible to a user of the dental tool substantially in real time, analyzing the video to determine a quantity of plaque at a plurality of locations on the plurality of teeth, determining, at a first time, that the quantity of plaque exceeds a threshold for an unclean location of the plurality of locations, superimposing a respective icon in the video at the unclean location such that the respective icon is visible to the user when the unclean location is visible to the user in the substantially real-time output, and determining, at a second time, that the quantity of plaque at the unclean location no longer exceeds the threshold and, in response, removing the icon from the superimposed video.

In an embodiment of the second aspect, the operations further include determining that a non-cleaning condition for an unclean location has been met and, in response, causing the icon associated with the unclean location to animate. In a further embodiment of the second aspect, the non-cleaning condition includes a predetermined passage of time after the first time or a passage of a working implement of the dental tool over the unclean location without sufficient removal of plaque.

In an embodiment of the second aspect, analyzing the video to determine a quantity of plaque at a plurality of locations on the plurality of teeth comprises detecting red fluorescence at each of the plurality of locations.

In an embodiment of the second aspect, the icon is a first icon, and removing the first icon from the superimposed video comprises replacing the first icon with a second icon.

In an embodiment of the second aspect, the operations further include analyzing the video to determine that a working implement of the dental tool is at the unclean location and, in response, animating the icon.

In a third aspect of the present disclosure, a method is provided that includes receiving a video of a user's mouth, the video captured by a dental tool comprising a camera and a cleaning implement, causing the video to be output to a display visible to a user of the dental tool substantially in real time, analyzing the video to determine a quantity of plaque at a first plurality of locations on a plurality of teeth in the mouth and determine a color of gum tissue at a second plurality of locations in the mouth. superimposing a respective icon in the video based on the determined quantity of plaque at an unclean location of the first plurality of locations such that the respective icon is visible to the user when the unclean location is visible to the user in the substantially real-time output, and determining that the color of gum tissue deviates from a previous color of the user's gum tissue by more than a threshold amount and, in response, outputting an indication of potential gum disease to the user on the display.

In an embodiment of the third aspect, analyzing the video is further to determine a respective location of each of the plurality of teeth with respect to adjacent teeth, and the method further includes determining that at least one of the teeth has moved with respect to a previous location of the at least one of the teeth and, in response, outputting an indication of a moved tooth to the user on the display.

In an embodiment of the third aspect, analyzing the video is further to determine a contour of the user's respiratory passageway, and the method further comprises determining that the contour is indicative of a respiratory problem and, in response, outputting an indication of a potential respiratory problem to the user on the display.

In an embodiment of the third aspect, the method further includes outputting an instruction for a user to expose the user's teeth and gums to a UV light of the dental tool.

In an embodiment of the third aspect, the method further includes transmitting a notification of potential gum disease to a care provider associated with the user. In a further embodiment of the third aspect, the method further includes receiving contact information for the care provider and receiving, from the user, a consent to share the user's personal medical information with the care provider.

In an embodiment of the third aspect, analyzing the video to determine a quantity of plaque at a plurality of locations on the plurality of teeth comprises detecting red fluorescence at each of the plurality of locations.

While this disclosure has described certain embodiments, it will be understood that the claims are not intended to be limited to these embodiments except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed embodiments. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

Some portions of the detailed descriptions of this disclosure have been presented in terms of procedures, logic blocks, processing, and other symbolic representations of operations on data bits within a computer or digital system memory. These descriptions and representations are the means used by those skilled in the data processing arts to most effectively convey the substance of their work to others skilled in the art. A procedure, logic block, process, etc., is herein, and generally, conceived to be a self-consistent sequence of steps or instructions leading to a desired result. The steps are those requiring physical manipulations of physical quantities. Usually, though not necessarily, these physical manipulations take the form of electrical or magnetic data capable of being stored, transferred, combined, compared, and otherwise manipulated in a computer system or similar electronic computing device. For reasons of convenience, and with reference to common usage, such data is referred to as bits, values, elements, symbols, characters, terms, numbers, or the like, with reference to various embodiments of the present invention.

It should be borne in mind, however, that these terms are to be interpreted as referencing physical manipulations and quantities and are merely convenient labels that should be interpreted further in view of terms commonly used in the art. Unless specifically stated otherwise, as apparent from the discussion herein, it is understood that throughout discussions of the present embodiment, discussions utilizing terms such as “determining” or “outputting” or “transmitting” or “recording” or “locating” or “storing” or “displaying” or “receiving” or “recognizing” or “utilizing” or “generating” or “providing” or “accessing” or “checking” or “notifying” or “delivering” or the like, refer to the action and processes of a computer system, or similar electronic computing device, that manipulates and transforms data. The data is represented as physical (electronic) quantities within the computer system's registers and memories and is transformed into other data similarly represented as physical quantities within the computer system memories or registers, or other such information storage, transmission, or display devices as described herein or otherwise understood to one of ordinary skill in the art.

What is claimed is:

1. A method for augmenting video from a dental tool, the method comprising:
receiving a video of a plurality of teeth, the video captured by a dental tool comprising a camera and a cleaning implement;
causing the video to be output to a display visible to a user of the dental tool substantially in real time;
analyzing the video to determine a quantity of plaque at a plurality of locations on the plurality of teeth;
determining, at a first time, that the quantity of plaque exceeds a threshold for an unclean location of the plurality of locations;
superimposing a respective icon in the video at the unclean location such that the respective icon is visible to the user when the unclean location is visible to the user in the substantially real-time output; and
determining, at a second time, that the quantity of plaque at the unclean location no longer exceeds the threshold and, in response, removing the icon from the superimposed video.

2. The method of claim 1, further comprising:
determining that a non-cleaning condition for an unclean location has been met and, in response, causing the icon associated with the unclean location to animate.

3. The method of claim 2, wherein the non-cleaning condition comprises:
a predetermined passage of time after the first time; or
a passage of a working implement of the dental tool over the unclean location without sufficient removal of plaque.

4. The method of claim 1, wherein analyzing the video to determine a quantity of plaque at a plurality of locations on the plurality of teeth comprises detecting red fluorescence at each of the plurality of locations.

5. The method of claim 1, wherein:
the icon is a first icon; and
removing the first icon from the superimposed video comprises replacing the first icon with a second icon.

6. The method of claim 1, further comprising:
analyzing the video to determine that a working implement of the dental tool is at the unclean location and, in response, animating the icon.

7. The method of claim 1, further comprising outputting an instruction for a user to expose the user's teeth to a UV light of the dental tool.

8. A dental tool system, comprising:
a dental tool comprising:
a camera;
a UV light; and
a working implement; and
a non-transitory, computer readable medium storing instructions that, when executed by a processor of a computing system, cause the computing system to perform operations comprising:
instructing a user, via a display, to irradiate the user's teeth using the UV light;
receiving a video of a plurality of teeth, the video captured by the dental tool camera;
causing the video to be output to a display visible to a user of the dental tool substantially in real time;
analyzing the video to determine a quantity of plaque at a plurality of locations on the plurality of teeth;
determining, at a first time, that the quantity of plaque exceeds a threshold for an unclean location of the plurality of locations;
superimposing a respective icon in the video at the unclean location such that the respective icon is visible to the user when the unclean location is visible to the user in the substantially real-time output; and
determining, at a second time, that the quantity of plaque at the unclean location no longer exceeds the threshold and, in response, removing the icon from the superimposed video.

9. The system of claim 8, wherein the operations further comprise:
determining that a non-cleaning condition for an unclean location has been met and, in response, causing the icon associated with the unclean location to animate.

10. The system of claim 9, wherein the non-cleaning condition comprises:
a predetermined passage of time after the first time; or
a passage of a working implement of the dental tool over the unclean location without sufficient removal of plaque.

11. The system of claim 8, wherein analyzing the video to determine a quantity of plaque at a plurality of locations on the plurality of teeth comprises detecting red fluorescence at each of the plurality of locations.

12. The system of claim 8, wherein:
the icon is a first icon; and
removing the first icon from the superimposed video comprises replacing the first icon with a second icon.

13. The system of claim 8, wherein the operations further comprise:
analyzing the video to determine that a working implement of the dental tool is at the unclean location and, in response, animating the icon.

14. A method comprising:
receiving a video of a user's mouth, the video captured by a dental tool comprising a camera and a cleaning implement;
causing the video to be output to a display visible to a user of the dental tool substantially in real time;
analyzing the video to:
determine a quantity of plaque at a first plurality of locations on a plurality of teeth in the mouth; and
determine a color of gum tissue at a second plurality of locations in the mouth;
superimposing a respective icon in the video based on the determined quantity of plaque at an unclean location of the first plurality of locations such that the respective icon is visible to the user when the unclean location is visible to the user in the substantially real-time output; and
determining that the color of gum tissue deviates from a previous color of the user's gum tissue by more than a threshold amount and, in response, outputting an indication of potential gum disease to the user on the display.

15. The method of claim 14, wherein:
analyzing the video is further to determine a respective location of each of the plurality of teeth with respect to adjacent teeth; and
the method further comprises determining that at least one of the teeth has moved with respect to a previous location of the at least one of the teeth and, in response, outputting an indication of a moved tooth to the user on the display.

16. The method of claim 14, wherein:
analyzing the video is further to determine a contour of the user's respiratory passageway; and
the method further comprises determining that the contour is indicative of a respiratory problem and, in response, outputting an indication of a potential respiratory problem to the user on the display.

17. The method of claim 14, further comprising outputting an instruction for a user to expose the user's teeth and gums to a UV light of the dental tool.

18. The method of claim 14, further comprising:
transmitting a notification of potential gum disease to a care provider associated with the user.

19. The method of claim 18, further comprising:
receiving contact information for the care provider; and
receiving, from the user, a consent to share the user's personal medical information with the care provider.

20. The method of claim 14, wherein analyzing the video to determine a quantity of plaque at a plurality of locations on the plurality of teeth comprises detecting red fluorescence at each of the plurality of locations.
